(19) United States
(12) Patent Application Publication
(10) Pub. No.: US 2013/0236897 A1
(43) Pub. Date: Sep. 12, 2013

(54) Anaplasma FAMILY MEMBERS CAUSE A NUMBER OF BLOOD-BORNE DISEASES

(71) Applicant: Charles A. Kallick, Seattle, WA (US)

(72) Inventor: Charles A. Kallick, Seattle, WA (US)

(21) Appl. No.: 13/416,827

(22) Filed: Mar. 9, 2012

(51) Int. Cl. C12Q 1/68

(52) U.S. Cl. CPC C12Q 1/689
USPC 435/6.12; 536/24.33

(57) ABSTRACT

A method of assaying for the presence of Anaplasma-infected blood cells or blood cell precursors from a patient is disclosed. The method comprises a) amplifying a nucleic acid-containing preparation of blood cells or blood precursor cells with a primer that contains a sequence of at least 12 consecutive bases of a polynucleotide of SEQ ID NOs:1-16 or an antisense sequence thereof; and detecting the presence of an amplified nucleic acid sequence. The presence of an amplified sequence indicates that the patient is suffering from Anaplasma-infected blood cells or blood cell precursors.

### TECHNICAL FIELD

The present invention relates to a gene of an organism that has been found in the blood and marrow cells of patients with a wide variety of diseases. The organism appears to be a member of the Anaplasma family.

### BACKGROUND ART

The genera Anaplasma, Ehrlichia, Cowdria, Neorickettsia and Wolbachia encompass a group of obligate intracellular bacteria that reside in vacuoles of eukaryotic cells and were previously placed in taxa based upon morphological, ecological, epidemiological and clinical characteristics. Recent genetic analyses of 16S rRNA genes, groESL and surface protein genes have indicated that the existing taxa designations are flawed. Dumler et al., IJSEM 2001 51:2145-2165.

Dumler et al. rearranged the taxa to yield four distinct clades characterized roughly as Anaplasma (including the Ehrlichia phagocytophylium group, Ehrlichia platys and Ehrlichia bovis) with a minimum of 96<1% similarity, Ehrlichia (including Cowdria ruminantium) with a minimum of 97<7% similarity, Wolbachia with a minimum of 95<6% similarity and Neorickettsia (including Ehrlichia sennetsu and Ehrlichia risticii) with a minimum of 94<9% similarity.

The resulting genus Anaplasma is extensively studied in veterinary medicine. The Anaplasma group of pathogens is sometimes referred to here as the “Ap group”. Members of the genus cause an economically important infection of the erythrocytes of cattle. Anaplasma infection of cattle produces a hemolytic anemia, economic losses, and an unusual syndrome of chronic rheumatoid illness in those animals considered too valuable for euthanization. The causative agent is Anaplasma marginale.

Anaplasma are tiny (0.2-2 μm) obligate, intracytoplasmic, gram-negative organisms that resemble Rickettsia; divide by binary fission; and multiply within the cytoplasm of infected white blood cells. Clusters of Anaplasma multiply in host monocyte vacuoles (phagosomes) to form large, mulberry-shaped aggregates called morulae.

Anaplasma inclusion bodies, such as morulae, are visible in the cytoplasm of infected mononuclear phagocytic cells after 5-7 days. The type of disease that develops varies and depends on the infecting species and the type of leukocyte infected.

Human granulocytic anaplasmosis (HGA), formerly known as human granulocytic ehrlichiosis (HGE), is caused by Anaplasma phagocytophylium, which infect granulocytes. In contrast, human monocytic ehrlichiosis (HME) now human monocytic Anaplasmosis (RMA), is caused by Ehrlichia chaffeensis, now Anaplasma chaffeensis, which infects monocytes.

Another Anaplasmal disease of canines was first reported in North Africa, but is worldwide in distribution. It can produce a carrier state, but in susceptible animals, it produces an illness called Tropical Canine Pancytopenia. This disease is essentially an intractable, progressive fatal aplastic anemia. Its causative agent was well studied during the Vietnam War, and has been named Ehrlichia canis. [Wandragula et al., “Anaplasmosis” in Woldehewit et al. Eds., Rickettsial and Chlamydial Diseases of Domestic Animals, Pergamon Press, Oxford, United Kingdom, 1993, Chapter 3, pages 65-88.]

These leukocyte-infecting parasitic bacteria are also known at times to alter host cell DNA during cell division. Ap accomplishes this survival strategy in part by eliciting an intracellular protein called host transcriptional protein (HTP). HTP does not kill the host cell, but may alter the nuclear instructions in cell development so that it fails to follow its programming. Early studies have confirmed a transcriptional response in cell division to infection with Anaplasma chaffeensis [See, Sukumaran et al., Infect Imm 2005 73:80989-80990; Zhang et al., Infect Imm 2004 72:498-507].

This unusual property of the Ap becomes important when host cells are in bone marrow and especially if they are in the stem cells. In that case, the alterations produced by Ap, mediated through HTP could force errors in transcription of proliferating cells and compromise the function of key immune system components. Thus, if stem cells were infected with an agent that harms their program of differentiation and development, the now abnormally functioning cell may be manifested and identified only as a later differentiated cell, including abnormally functioning T or B cells.

If the stem cell is infected by an Ap or similar agent, the persisting self-reactive B cell may not be eliminated by the marrow mechanisms and could by chance be reactive against myelin. This result could steadily damage the myelin of the peripheral nerves, leading to progressive loss of function. This hypothesis offers a plausible model for the development of multiple sclerosis (MS). Similarly, abnormal T cells directed against a component of the anterior horn cells in the spinal cord could be damaged by constant activity of self-reactive T cells from a small number of infected stem cells in amyotrophic lateral sclerosis (ALS). Infection of marrow stem cells may affect one type of immune cell, but spreading of infection to other stem cells and then to other marrow cells could reduce the ability of the marrow to produce the cells needed by the body, especially the leukocytes and megakaryocytes. This is a model similar to end stage marrow disease in leukemia.

Human disease associated with a presumed Ehrlichia (Anaplasma) was reported in a patient with idiopathic aplastic anemia in 1973. It was the first report of human infection with an Ehrlichial (Anaplasmal) agent [Kallick et al., “Human Bone marrow failure associated with Ehrlichia canis”, Presented at the Interscience Conference on Antimicrobial Agents and Chemotherapy, Washington D.C., 1973, Abstract #1].

Later reports of several Anaplasmal agents associated with tick transmission have been described, and the resulting diseases derive their names from the characteristic blood cell parasitized. The best studied of these diseases is Human Granulocytic Anaplasmosis (HGA). As the name implies, it is found in the peripheral circulation in granulocytes. The infective agent was originally called an Ehrlichia, but following the isolation of the causative agent, and molecular investigation, it was renamed Anaplasma phagocytophylium. [Dumler et al., J. Infect Disease 1996 173:1027-1030].

Klein et al., Antimicrob. Agents and Chemother., January 1997 41(1):76-79, reported a study of several drugs against the HGA agent. Of the active agents assayed, those authors reported that doxycycline, rifampin, rifabutin, ciprofloxacin, ofloxacin and trovafloxacin were the most active, and bactericidal, with the HGA agent being resistant to clindamycin, thrmethoprim-sulfamethoxazolem imipenem-cilastatin, ampicillin, cefatriaxone, erythromycin and azrithromycin. Gentamicin and chloramphenicol were weakly active, but not bactericidal.

An association of a Haemobartonella-like organisms with systemic lupus erythematosus (SLE) was reported in 1972 [Kallick et al., Nature, New Biology 1972 236:145-146], and an antigenic relationship of SLE to Anaplasma marginale (AM) was confirmed in 1980 [Kallick et al., Arthr. and Rheumat., 1980 23:197-205]. In that study, all of 22 patients with SLE had antibodies to AM. Evidence of deposits of antigen was also seen by fluorescent antibody in the glomeruli of a patient with lupus nephritis. Continued study by many investigators has established that most of these reported organisms are related antigenically [Casperson et al., Infect. and Immuni. 2002 70:1230-1234].

The single case of human aplastic anemia of interest here was reported in 1973 and involved a 52 year old Caucasian man, who suffered from idiopathic aplastic anemia [Kallick et al., “Human Bone marrow failure associated with Ehrlichia canis”, Presented at the Interscience Conference on Antimicrobial Agents and Chemotherapy, Washington D.C., 1973, Abstract #1]. Because of some clinical similarities to the canine syndrome of Tropical Canine Pancytopenia, this patient's blood was examined by a technique developed by (Nyindo et al., Amer. J. Vet. Res. 1971 32:1651-1658). Although not used today, the technique involved use of the patient's own harvested monocytes. When separated and fed in a culture medium, the monocytes cling to an immersed glass slide. When fed with medium for 2 weeks, the monocytes, now macrophages, exhibited intracytoplasmic inclusions like morulae that stained with antibody to E. canis (now A. canis; FIG. 2).

The supernatant from that culture method was injected into a rhesus monkey, whose monocytes had been previously examined similarly for evidence of antigenically stained inclusions and found to be lacking such evidence. After several weeks of incubation, the monkey's macrophages had changed to exhibit similarly stained inclusions (FIG. 3) [Kallick et al., “Human Bone marrow failure associated with Ehrlichia canis”, Presented at the Interscience Conference on Antimicrobial Agents and Chemotherapy, Washington D.C., 1973, Abstract #1].

The invention below describes a portion of a gene that can be used to identify the bacterial organism of the above infections.

### BRIEF SUMMARY OF THE INVENTION

The present invention contemplates a polynucleotide and a method of its use to identify the presence of blood- or marrow-borne infecting organisms. Infected blood and/or marrow cells contain inclusions that themselves contain nucleic acid that can be amplified using a primer of SEQ ID NOs:1-16. Those infected cells also typically immunoreact with anti-E. canis antibodies, and contain inclusions that stain with acridine orange or giemsa and are also visible in phase contrast microscopy once stained.

More particularly, the present invention contemplates a method of assaying for the presence of Anaplasma-infected blood cells or blood cell precursor cells of a patient. The method comprises amplifying a nucleic acid-containing preparation of blood cells or blood precursor cells from a patient with a primer that contains a sequence of at least 12 consecutive bases of a polynucleotide of SEQ ID NOs:1-16, or a complementary sequence thereof (an antisense sequence thereof). A primer of one or more of SEQ ID NOs:10-16, or its antisense sequence is particularly preferred. The presence of an amplified nucleic acid sequence is then detected. The presence of an amplified sequence indicates that the patient suffers from Anaplasma-infected blood cells or blood cell precursors.

Preferably, the primer is double-stranded. It is also preferred that the primer contain a sequence of about 20 to about 40 consecutive bases of a polynucleotide of SEQ ID NOs:1-16 or its antisense sequence, and more preferably, about 20 to about 30 consecutive bases. A patient whose blood or blood precursor cells are assayed typically exhibits symptoms of systemic lupus erythematosus, aplastic anemia, bladder cancer, or a leukemia.

### BRIEF DESCRIPTION OF THE DRAWINGS

In the drawings forming a part of this invention,

FIG. 1, adapted from Iwasaki et al., Immunology 2003 19:451-462 is a schematic depiction of hematopoietic cell differentiation in which HSC=Human Stem Cell, CMP=myelomocytic progenitor, CLP=Lymphoid progenitor, GMP=granulocyte-monocyte progenitor, MEP=erythrocyte-megakaryocyte progenitor, and ProT=Lymphocyte T cell progenitor, and ProB=lymphocyte B cell progenitor.

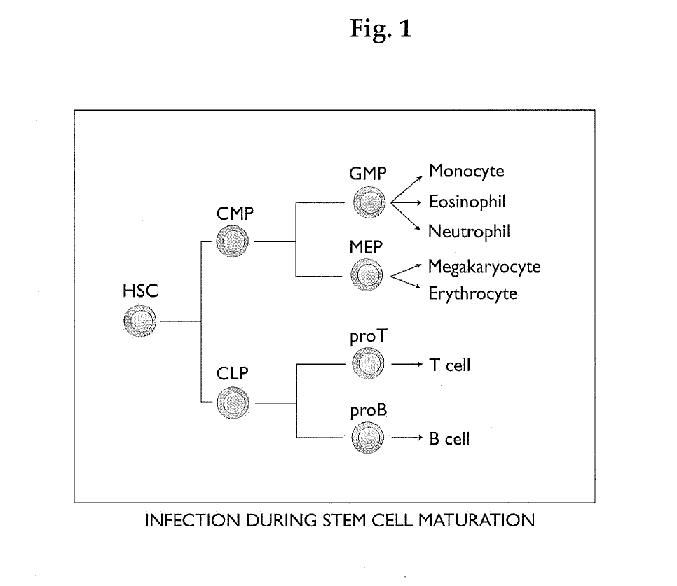

FIG. 2 is a photomicrograph of a macrophage from a patient with aplastic anemia that was stained with a direct conjugate of anti-E. canis antibodies (Courtesy of Dr. M Nyindo and the late Dr. M. Ristic). A circulating cell from a patient with acute myelocytic leukemia. Staining is with direct reacting anti-A. canis 1:4. The light areas in this circulating blood cell represent the bright staining of anti-A. canis which delineate several areas. The upper rod like structure resembles an Auer rod seen in myelocytic leukemias [Auer, Am J Med Sci 1906 131:1002-1015], and the two bright areas at the bottom of the cell appear to resemble morulae of developing Anaplasmae. The concentration of anti-A. canis used by Dr. Nyindo who conducted the laboratory work in this incident, was to compensate for the observed cross reactivity of a likely related Ap to the presumed one in this patient.

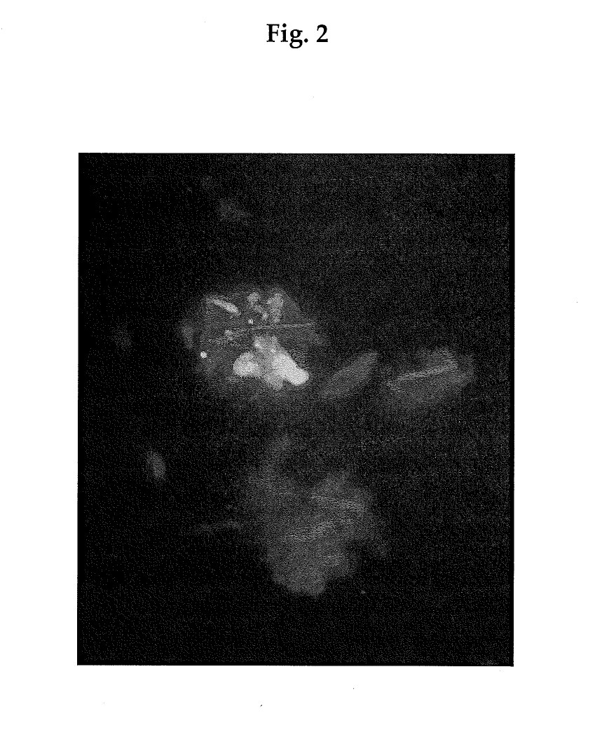

FIG. 3 is a photomicrograph of a Rhesus monkey monocyte removed from the animal after injection of culture medium supernatant from the cells of a human with aplastic anemia in to the animal and maintenance of the animal for several weeks.

FIG. 4 is a photograph of a gel of nucleic acid cloned by PCR technology from the blood of a patient diagnosed as having an aggressive bladder cancer. Specifics of this study are discussed hereinafter.

### DEFINITIONS

As used herein, the 3′-terminal region of the nucleic acid probe refers to the region of the probe including nucleotides within about 10 residues from the 3′-terminal position.

In either a linear DNA molecule, discrete elements are referred to as being “upstream” or “5′” relative to an element if they are bonded or would be bonded to the 5′-end of that element. Similarly, discrete elements are “downstream” or “3′” relative to an element if they are or would be bonded to the 3′-end of that element. Transcription proceeds in a 5′ to 3′ manner along the DNA strand. This means that RNA is made by the sequential addition of ribonucleotide-5′-triphosphates to the 3′-terminus of the growing chain (with the elimination of pyrophosphate).

As used herein, the term “amplifying” or “amplification” means the production of additional copies of the DNA sequence sought (target nucleic acid) and is generally carried out using polymerase chain reaction (PCR) or other technologies well known in the art. With PCR, it is possible to amplify a single copy of a sought-after DNA sequence to a level that can be detected by several different methodologies.

As used herein, and unless otherwise indicated, the term “antisense oligonucleotide” refers to an oligonucleotide having a sequence complementary to a target DNA or RNA sequence.

The term “immunoreact” is used to mean a specific binding between an antigenic determinant-containing molecule and a molecule containing an antibody combining site such as a whole antibody molecule or a portion thereof.

As used herein the word “antibody” refers to immunoglobulin molecules and immunologically active or functional fragments of immunoglobulin molecules that contain an antigen recognition and binding site. Exemplary antibody molecules are intact immunoglobulin molecules, substantially intact immunoglobulin molecules and active fragments of an immunoglobulin molecule, including those portions known in the art as Fab, Fab′, F(ab′)₂ and scFv.

As used herein, the term “target nucleic acid” or “nucleic acid target” refers to a particular nucleic acid sequence of interest. Thus, the “target” can exist in the presence of other nucleic acid molecules or within a larger nucleic acid molecule.

As used herein, the term “nucleic acid probe” refers to an oligonucleotide or polynucleotide that is capable of hybridizing to another nucleic acid of interest. A nucleic acid probe can occur naturally as in a purified restriction digest or be produced synthetically, recombinantly or by PCR amplification. As used herein, the term “nucleic acid probe” refers to the oligonucleotide or polynucleotide used in a method of the present invention. That same oligonucleotide could also be used, for example, in a PCR method as a primer for polymerization, but as used herein, that oligonucleotide would then be usually referred to as a “primer”.

As used herein, the term “primer” refers to an oligonucleotide, whether occurring naturally as in a purified restriction digest or produced synthetically, that is capable of acting as a point of initiation of synthesis when placed under conditions in which synthesis of a primer extension product that is complementary to a nucleic acid strand is induced, (e.g., in the presence of nucleotides and of an inducing agent such as DNA polymerase and at a suitable temperature and pH). The primer is preferably single stranded for maximum efficiency in amplification, but can alternatively be double stranded. If double stranded, the primer is first treated to separate its strands before being used to prepare extension products. Preferably, the primer is an oligodeoxyribonucleotide.

The primer must be sufficiently long to prime the synthesis of extension products in the presence of the inducing agent. The exact lengths of the primers will depend on many factors, including temperature, source of primer and the use of the method. A primer typically contains a sequence of at least about 12, preferably about 25, more preferably about 40, 50 or 75 consecutive nucleotides of the sequence to be amplified.

As used herein, the terms “complementary” or “complementarity” are used in reference to nucleic acids (i.e., a sequence of nucleotides) related by the well-known base-pairing rules that A pairs with T and C pairs with G. For example, the sequence 5′-A-G-T-3′, is complementary to the sequence 3′-T-C-A-5′. Complementarity can be “partial,” in which only some of the nucleic acid bases are matched according to the base pairing rules. On the other hand, there may be “complete” or “total” complementarity between the nucleic acid strands when all of the bases are matched according to base pairing rules. The degree of complementarity between nucleic acid strands has significant effects on the efficiency and strength of hybridization between nucleic acid strands as known well in the art. This is of particular importance in detection methods that depend upon binding between nucleic acids, such as those of the invention. The term “substantially complementary” refers to any probe that can hybridize to either or both strands of the target nucleic acid sequence under conditions of low stringency as described below or, preferably, in polymerase reaction buffer (e.g., Promega, M195A) heated to 95° C. and then cooled to room temperature. As used herein, when the nucleic acid probe is referred to as partially or totally complementary to the target nucleic acid, that refers to the 3′-terminal region of the probe (i.e. within about 10 nucleotides of the 3′-terminal nucleotide position).

As used herein, the term “hybridization” is used in reference to the pairing of complementary nucleic acid strands. Hybridization and the strength of hybridization (i.e., the strength of the association between nucleic acid strands) is impacted by many factors well known in the art including the degree of complementarity between the nucleic acids, stringency of the conditions involved affected by such conditions as the concentration of salts, the Tₘ (melting temperature) of the formed hybrid, the presence of other components (e.g., the presence or absence of polyethylene glycol), the molarity of the hybridizing strands and the G:C content of the nucleic acid strands.

As used herein, the term “stringency” is used in reference to the conditions of temperature, ionic strength, and the presence of other compounds, under which nucleic acid hybridizations are conducted. With “high stringency” conditions, nucleic acid base pairing will occur only between nucleic acid fragments that have a high frequency of complementary base sequences. Thus, conditions of “weak” or “low” stringency are often required when it is desired that nucleic acids that are not completely complementary to one another be hybridized or annealed together. The art knows well that numerous equivalent conditions can be employed to comprise low stringency conditions. However, illustrative high, moderate and low stringency conditions are set out below.

Polynucleotide hybridization is a function of sequence identity (homology), G+C content of the sequence, buffer salt content, sequence length and duplex melt temperature (Tₘ) among other variables. See, Maniatis et al., Molecular Cloning, Cold Spring Harbor Laboratory, Cold Spring Harbor, N.Y. (1982), page 388.

With similar sequence lengths, the buffer salt concentration and temperature provide useful variables for assessing sequence identity (homology) by hybridization techniques. For example, where there is at least 90 percent homology, hybridization is carried out at 68° C. in a buffer salt such as 6× sodium chloride/sodium citrate (SCC) diluted from 20×SSC [Maniatis et al., above, at page 447] and two sequences form a hybrid duplex (hybridize). The buffer salt utilized for final Southern blot washes can be used at a low concentration, e.g., 0.1×SSC and at a relatively high temperature, e.g. 68° C. Use of the above hybridization and washing conditions together are defined as conditions of high stringency or highly stringent conditions.

Moderately high stringency conditions can be utilized for hybridization where two sequences share at least about 80 percent homology. Here, hybridization is carried out using 6×SSC at a temperature of about 50-55° C. A final wash salt concentration of about 1-3×SSC and at a temperature of about 60-68° C. are used. These hybridization and washing conditions define moderately high stringency conditions.

Low stringency conditions can be utilized for hybridization where two sequences share at least 30 and more preferably about at least 40 percent homology. Here, hybridization carried out using 6×SSC at a temperature of about 40-50° C., and a final wash buffer salt concentration of about 6×SSC used at a temperature of about 40-60° C. effect non-random hybridization. These hybridization and washing conditions define low stringency conditions.

As used herein, the term “Tₘ” is used in reference to the “melting temperature”. The melting temperature is the temperature at which 50% of a population of double-stranded nucleic acid molecules becomes dissociated into single strands. The equation for calculating the Tₘ of nucleic acids is well-known in the art. The Tₘ of a hybrid nucleic acid is often estimated using a formula adopted from hybridization assays in 1 M salt, and commonly used for calculating Tₘ for PCR primers: {[(number of A+T) X2° C.+(number of G+C) X 4° C.]} Newton et al. PCR, 2.sup.nd Ed., Springer-Verlag (New York: 1997), p. 24. This formula was found to be inaccurate for primers longer that 20 nucleotides. Id. Other more sophisticated computations exist in the art that take structural as well as sequence characteristics into account for the calculation of Tₘ. A calculated Tₘ value is merely an estimate; the optimum temperature is commonly determined empirically.

A melt curve can also used to determine the degree of likeness among amplified fragments by the base pair make-up; i.e., if the fragments are 100% the same as far as A/T and G/C content are concerned, they will melt at the same temperature. If they vary from one another, then they melt at different temperatures. This method takes advantage of the two hydrogen bonds that allow A's and T's to base pair and 3 that a lot for the G/C base pairing; the G/C base pairs take more heat/energy to break the bonds and disrupt the double-stranded DNA because of the extra hydrogen bond they form.

The term “homology,” as used herein, refers to a degree of complementarity. There can be partial homology or complete homology (i.e., identity). A partially complementary sequence that at least partially inhibits a completely complementary sequence from hybridizing to a target nucleic acid is referred to using the functional term “substantially homologous.”

When used in reference to a double-stranded nucleic acid sequence such as a cDNA or genomic clone, the term “substantially homologous,” as used herein, refers to a probe that can hybridize to a strand of the double-stranded nucleic acid sequence under conditions of low stringency.

The terms “subject” or “patient” are used interchangeably herein, and is meant a mammalian subject to be treated, with human subjects being preferred. In some cases, a method of the invention finds use in experimental animals, in veterinary application, and in the development of animal models for disease, including, but not limited to, rodents including mice, rats, and hamsters; companion animals such as dogs, cats, horses, livestock animals such as cattle, swine, sheep, goats, and primates such as man, gorillas, chimpanzees, gibbons, orangutans, and monkeys such as green monkeys, cynomolgus monkeys or macaques.

“Substantially purified” refers to nucleic acid molecules that are removed from their natural environment and are isolated or separated, and are at least about 60% free, preferably about 75% free, and most preferably about 90% free, from other components with which they are naturally associated.

### DETAILED DESCRIPTION OF THE INVENTION

The present invention contemplates a method of assaying for the presence of Anaplasma-infected blood cells or blood cell precursor of a patient. The method comprises amplifying a nucleic acid-containing preparation of blood cells or blood precursor cells from a patient with a primer that contains a sequence of at least 12 consecutive bases of a polynucleotide of SEQ ID NOs:1-16, or a complementary sequence thereof. The presence of an amplified nucleic acid sequence is then detected. Detection of amplified nucleic acid sequences is well known and illustrated hereinafter using gel electrophoresis. The presence of an amplified sequence indicates that the patient suffers from Anaplasma-infected blood cells or blood cell precursors.

A contemplated primer preferably contains a sequence of about 20 to about 40 consecutive bases, and more preferably about 20 to about 30 consecutive bases. In other embodiments, a primer contains about 40, 50 or 75 consecutive nucleotides of a sense strand of a sequence set forth in SEQ ID NOs:1-16, an anti-sense sequence (complement) of a sequence set forth in SEQ ID NO:1-16 or a double-stranded sequence of an above length. A contemplated primer is preferably a synthetic polynucleotide that is isolated and purified prior to use. A particularly preferred primer has the sequence of SEQ ID NOs:10-16.

A patient from whom the blood cells or blood precursor cells are prepared typically exhibits symptoms of systemic lupus erythematosus, aplastic anemia, bladder cancer, or leukemia. A leukemia of which a patient exhibits symptoms can be acute lymphocytic leukemia, acute myelogenous leukemia, polycythemia vera or chronic lymphoid leukemia, among others.

### The Infectious Bacterium

The SLE-causing infectious bacterium infection present in vivo can be detected in vitro by PCR using primers designed specifically to amplify only Ap group DNA as well as with SEQ ID NO: 4. PCR can be conducted on isolated infected cell culture DNA.

Primers are designed based upon the DNA sequence of the 16S ribosomal RNA gene in regions conserved among A. equi, A. phagocytophylium, and the HGA agent. Primers are typically complementary to and/or hybridize to a 5′ region and a 3′ region of the nucleic acid sequence. Primers preferably have at least 15 nucleotides that are complementary to the nucleic acid sequence. Primers are preferably about 15 to about 50 nucleotides long and can be prepared by automated synthesis.

Two sets of preferred useful primers (SLE1-F, SLE1-R, SLE2 and SLE3) amplify a 919 base pair fragment of the Ap group 16S ribosomal RNA gene and have the following sequences [Chen et al, J. Clin. Microbiol., 32:589 (1994)] and U.S. Pat. No. 5,955,359:

SLE1-f
(SEQ ID NO: 1)
5′-AACGGATTATTCTTTATAGCTTGCT-3′;
SLE1-r
(SEQ ID NO: 2)
5′-GGAGATTAGATCCTTCTTAACGGAA-3′;

or a 928 base pair fragment of the Ap group 16S ribosomal RNA gene and have the following sequences:

SLE2
(SEQ ID NO: 3)
5′-GTCGAACGGATTATTCTTTATAGCTTGC-3′,
SLE3
(SEQ ID NO: 4)
5′-CCCTTCCGTTAAGAAGGATCTAATCTCC-3′.

A further useful sequence obtained from an SLE patient's blood is designated SLE4 and has the sequence:

(SEQ ID NO: 5)
5′-TCGTAACCAA TCTCAAGCTC AACCCTGGCA
CCACCAATAC CATAACCAAC ACTGCCTTCC ATAGCTACAA
GCATGTTGTC CTTAAACCCA ATCCGAGGAT CAGGAGTGTT
CCAGTCA

and can be used in hybridization assays.

Alternately, the following primers can be constructed to specifically amplify a 150 bp DNA fragment from A. equi and Ap group Anaplasmae as discussed for other purposes in U.S. Pat. No. 5,928,879. Examples of such primers include:

SLE5
(SEQ ID NO: 6)
5′-TAGATCCTTCTTAACGGAAGGGCG-3′;
SLE6
(SEQ ID NO: 7)
5′-AAGTGCCCGGCTTAACCCGCTGGC-3′;
SLE7
(SEQ ID NO: 8)
5′-TTTATCGCTATTAGATGAGCCTATG-3′;
and
SLE8
(SEQ ID NO: 9)
5′-CTCTACACTAGGAATTCCGCTAT-3′.

A still further primer was found useful in identifying a portion of the gene of the Anaplasma that infected the blood of a patient with a myelodysplastic syndrome, severe poycythemia vera, and subsequent acute rapidly fatal myelocytic leukemia. That gene portion was first published in Kallick, Medical Hypotheses 2011 77:374-379 and is illustrated in SEQ ID NO: 10, with a useful primer therefore being illustrated in the SEQ ID NO:11, below.

SEQ ID NO: 10
TTTCGTAACC AATCTCAAGC TCAACCCWGG CACCACCAAT
ACCATAACCA ACACTGCCTT CCATAGCTAC AAGCATGTTG
TCCTTAAACC CAATCCGAGG ATCAGGAGTG TTCCAGTCAA
AGGGCGAATT CCAGCACAC,
where W = T or A
Primer
(SEQ ID NO: 11)
5′-TCCTCGGATTGGGTTTAAGGA

Further illustrative primer sequences are shown below:

Primer
SEQ ID NO: 12
5′-GCTTGAGATT GGTTACGAAA
Primer
SEQ ID NO: 13
5′-CCTGGGTTGA GCTTGAGATT GGTTA
Primer
SEQ ID NO: 14
5′-CCAGGGTTGA GCTTGAGATT GGTTA
Primer
SEQ ID NO: 15
5′-TGACTGGAAC ACTCCTGAT
Primer
SEQ ID NO: 16
5′-CCTCGGATTG GGTTTAAGGA CAACATGCTT GTAGCTATGG

A portion of the reported sequence that encodes the major surface protein of human granulocytic anaplasmosis (HGA) is shown in SEQ ID NO: 17, below.

SEQ ID NO: 17
HGA
TCGTAACC AATCTCAAGC TCAACCCTGG CACCACCAAT
ACCATAACCA ACACTGCCTT CCATAGCTAC AAGCATGTTG
TCCTTAAACC CAATCCGAGG ATCAGGAGTG TTCCAGTCA

In a preferred embodiment of the invention, the amplification is carried out by real-time quantitative PCR (Q-PCR). As used herein, the term “real-time quantitative PCR” or “Q-PCR” refers to a method based on the continuous optical monitoring of the progress of a fluorogenic PCR reaction. In this system, in addition to a pair of amplification primers used in conventional PCR, a dual-labeled fluorogenic hybridization probe is also included. One fluorescent dye serves as a reporter (e.g. FAM), and its emission spectra is quenched by a second fluorescent dye (e.g. TAMRA). During the extension phase of PCR, the 5′ to 3′ exonuclease activity of Taq DNA polymerase cleaves the reporter from the probe, thus releasing it from the quencher and resulting in an increase in fluorescence. The fluorescence is then used to monitor the amplification process and determine the amount of the original template DNA.

In some preferred embodiments, a kit for performing an assay using a method of the invention is contemplated. A kit according to the present invention comprises containers such as vials or ampules that each hold one or more primers as recited above. If desired, a kit can also comprise instructions for using the kit. Typically, the second unit of a kit further comprises a probe for a Q-PCR assay. In order to eliminate the false positive cases mentioned above, a kit preferably comprises primers for amplicons of different sizes. For example, a kit includes a pair of primers for amplifying an Anaplasma DNA sequence of about 100 bp and another pair of primers for amplifying a DNA sequence of 75 bp.

It should also be apparent to those skilled in the art that a contemplated kit can also be used to detect and/or quantify an Anaplasma RNA sequence in a sample preparation by using a reverse transcriptase polymerase chain reaction (RT-PCR) assay or a real-time quantitative reverse transcriptase polymerase chain reaction (Q-RT-PCR) assay, for the diagnosis, prognosis or monitoring of an Anaplasma-associated infection in an individual.

The method for extracting DNA fragments from plasma and cells is known in the art, e.g., the method by using a QIAamp® Blood Kit (Qiagen, Hilden, Germany) described by Lo et al., Cancer Res. 60(24):6878-6881 (2000). In brief, plasma samples are harvested from the patients according to established protocols in the art. The samples are stored at −20° C. until further processing. DNA from plasma samples is extracted using a QIAamp® Blood Kit (Qiagen, Hilden, Germany) using the “blood and body fluid protocol” as recommended by the manufacturer. A total of 400-800 μl of the plasma samples are used for DNA extraction per column. The exact amount is documented for the calculation of the target DNA concentration. A final elution volume of 50 μl is used to elute the DNA from the extraction column.

Once a patient is determined to have an Anaplasma infection, a treatment method is contemplated that comprises administering to the patient an antibacterial agent that is (i) doxycycline, (ii) an antibacterial amount of a rifamycin, (iii) an antibacterial amount of a quinolone, or a mixture of any of (i), (ii) and (iii). In preferred practice, the rifamycin is one or more of rifampin, rifabutin or rifamide, and the quinolone compound is selected from one or more of the group consisting of ciprofloxacin, levofloxacin, moxifloxacin, gatifloxacin, tefloxacin and trovafloxacin. The antibacterial agent is administered in an amount that is an antibacterial amount for that drug. In preferred practice, the antibacterial agent administration is repeated periodically until the bacterial structures are eliminated from the patient's marrow cells and/or erythrocytes or both.

Doxycycline is a semi-synthetic tetracycline drug that is used for treating a variety of infectious agents. The structural formula for doxycycline is shown below.

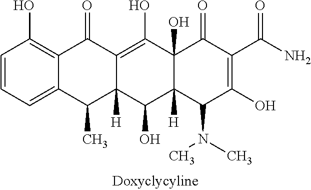

The phrase “a rifamycin” is meant to include rifamycin itself as well as rifamycin derivatives as are discussed below. Rifamycin is a broad-spectrum antibiotic produced by Streptomyces mediterranei that is active against most gram-positive organisms and has variable activity against gram-negative organisms such as Escherichia coli and Pseudomonas. Rifamycin and its derivatives also have intracellular bactericidal activity.

Rifampicin, or rifampin, whose structure is shown below, is a drug whose use has been approved

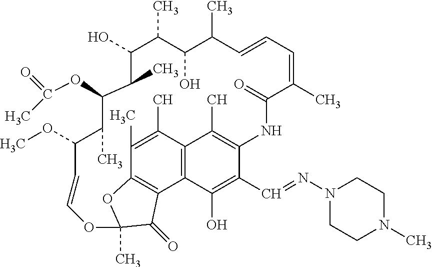

for treating infections caused by a great number of agents. Its action appears to be an effect on DNA-dependent RNA polymerase activity of bacterial infections. For example, rifampicin is useful for treating gram negative bacteria such as Neiseria meningitides, and M. tuberculosis.

Rifampicin is also effective against intracellular bacterial pathogens. Rifampicin is particularly effective against intracellular organisms in the genus of Ehrlich/Anaplasma, for which it appears to be the only effective antibiotic, with tetracycline being less effective and bacteriostatic.

Rifampin is a particularly preferred rifamycin derivative that is available from Aventis as RIFADIN®, and can administered per orally or by injection in an antibacterial amount. Capsules for oral administration are available that contain 150 or 300 mg of rifampin per capsule. Usual adult oral administrations are 600 mg once per day, usually with water about two hour before a meal, with dosages of about 450 to about 900 mg per day being contemplated. Combination therapeutics sold by Aventis under the name RIFATER® and RIFAMATE® can also be used. RIFATER® contains rifampin (120 mg), isoniazid (50 mg) and pyrazinamide (300 mg). RIFANATE® is a combination of isoniazid (150 mg) and rifampin (300 mg). These combination drugs are usually used to treat tuberculosis.

Rifabutin, available under the trademark MYCOBUTIN® from Pharmacia-Upjohn (now Pfizer), is also a preferred rifamycin derivative. Rifapentine, available from Aventis under the trademark PRIFTIN®, and rifamide are other rifamycin derivatives that can also be used.

It is to be understood that the rifampin need not be administered via the above-noted commercially available forms. Rather, those drugs can be compounded into a composition for administration to a SLE patient using well-known pharmaceutical techniques.

In another aspect of the invention, the leukemia or aplastic anemia patient is treated by administration of an antibacterial agent that is comprised of an antibacterial amount of a quinolone. Quinolone compounds typically contain two fused 6-membered rings that are aromatic and include at least one ring nitrogen atom having a keto group directly across the ring. A generic structure of the quinolone drugs is shown below, where Z can be carbon

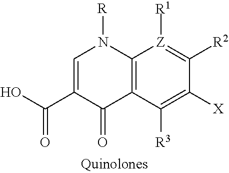

or nitrogen, X is fluoro in second and third generation drugs and the other R groups can be quite varied.

The first quinolone, nalidixic acid, contains two ring nitrogen atoms, is effective against Gram (−) bacteria. Nalidixic acid was first marketed in 1965 and is still available.

The quinolones are divided into generations based on their antibacterial spectrum. The earlier generation agents exhibit, in general, a narrower spectrum of activity than the later ones. As will be noticed from the list below, several quinolones have been withdrawn from the market. These withdrawals have been because of adverse side effects. However, in view of the lethality of the diseases discussed herein, the prescribing physician can elect to treat using a pharmaceutical that has been withdrawn from general usage.

Quinolone Generations

1st Generation
cinoxacin
nalidixic acid
oxolinic acid
piromidic acid
pipemidic acid
rosoxacin
2nd Generation
ciprofloxacin
enoxacin
fleroxacin (withdrawn)
lomefloxacin
nadifloxacin
norfloxacin
ofloxacin
pefloxacin
rufloxacin
3rd generation
balofloxacin
gatifloxacin (withdrawn)
grepafloxacin (withdrawn)
levofloxacin
moxifloxacin
pazufloxacin
sparfloxacin
temafloxacin (withdrawn)
4th generation
clinafloxacin
garenoxacin
gemifloxacin
prulifloxacin
sitafloxacin
trovafloxacin (withdrawn)

Ciprofloxacin (cipro) is a second generation drug within the quinolone class, specifically the fluoroquinolones. It is used to treat bacterial infections in various parts of the body when given orally and is a preferred quinolone for use herein. Use of a later generation quinolone is preferred over the use of an earlier generation drug (fourth over third generation, third over second generation, and second over first generation). Use of a floroquinolone is particularly preferred.

One way to assess how long to continue administration of an above-noted drug is to continue administration until the stainable, phase contrast microscopically visible, exogenous bacterial structures seen in the patient's erythrocytes and/or marrow cells prior to treatment are absent from those cells post treatment, and preferably until no further evidence of infection is present. This duration of administration can take as long as about 120 days, the average lifetime of an erythrocyte, or more. Periodically repeated administrations of drugs that encompass the average lifetime of an erythrocyte are thus contemplated and are preferred.

Probenecid, a drug that decreases renal excretion of some drugs such as those contemplated for use herein, can also be administered in conjunction with a before-mentioned antibacterial agent to provide its decreased renal excretion effect. It is to be understood that the antibacterial agent such as probenecid and rifampin need not be administered via the above-noted commercially available forms. Rather, those drugs can be compounded into a composition for administration to a leukemia or aplastic anemia patient using well-known pharmaceutical techniques.

### Example 1

### Preparation of Ehrlichia-Infected Cells

Following the teachings of Dumler et al., U.S. Pat. No. 5,955,359, Ap group granulocytic Anaplasma such as Anaplasma equi obtained from infected horses or the human granulocytic Anaplasma obtained from human patients with human granulocytic Anaplasmosis can be grown in promyelocyte cell cultures. Thus, Ap group granulocytic Anaplasmae, such as A. equi or the human granulocytic Anaplasma, are incubated with promyelocyte cell line cultures, preferably HL60 or KG-1 cell cultures. The A. equi and human granulocytic Anaplasma inoculum can be obtained from horses after experimental inoculation with thawed, A. equi- or human granulocytic Anaplasma-infected blood from an acutely infected horse, followed by venipuncture and removal of fresh, infected blood at a time when Anaplasmae are visible in the peripheral blood leukocytes of the ill, infected horse. Alternately, the human granulocytic Anaplasma inoculum can be obtained from human patients during the acute phase of human granulocytic ehrlichiosis at a time when typical Anaplasma morulae are detected within leukocytes in the peripheral blood of the infected patient.

In accordance with Dumler et al. U.S. Pat. No. 5,928,879, Ehrlichia equi (Anaplasma equi) obtained from infected horses can be grown in I. scapularis tick cell culture. In a method of that patent, Ap group granulocytic Anaplasmae, such as A. equi, are incubated with I. scapularis tick cell culture, preferably the IDE8 line [deposited with the American Type Culture Collection in Rockville, Md. on Aug. 26, 1995, under ATCC No. CRL 11973].

Tick cell cultures in which about 10% to about 50% of the cells are infected can be mixed with uninfected tick cells at a ratio of about 1:3 to about 1:20. Anaplasma equi is cultured in I. scapularis cells derived from a suspected natural vector of the pathogen. The requirements for successful isolation of the Anaplasmae in tick cell culture are: an atmosphere that is reduced in O₂ tension (from 20% O₂ as found in normal air to about 17%), and enhanced 10-fold with respect to CO₂ tension [from 0.03% as found in normal air to about 3%). The culture medium used also must include CO₂ in the form of NaHCO₃ (0.25% (w/v)], and an organic buffer. An organic buffer, such as 3-(N-morpholino)-propane sulfonic acid (MOPS) or N-(2-hydroxyethyl) piperazine-N-(2-ethanesulfonic acid) (HEPES), at a concentration of 10 to 15 mM is preferably included to ensure growth of A. equi.

The infected tick cultures are incubated at about 31° C. to about 35° C. until growth of E. equi is detected. Anaplasma equi grows to high yield in IDE8 cells as compared to neutrophilic granulocytes harvested from horses. Individual cells can contain enormous masses (>1,000) of Anaplasmae.

The organisms found in the neutrophils are contained within a membrane bound vacuole up to about 3 to 5 μm in diameter. The individual organisms are small, highly pleomorphic, and approximately 0.5 μm in size. With Romanowsky stains, such as Giemsa or LeukoStat®, Anaplasma species and the morulae appear bluish-purple. By electron microscopy their electron density is similar to that of the host cytoplasm, but some morulae may contain smaller, dense forms.

Illustratively, Ap group granulocytic Anaplasmae, such as A. equi or the human granulocytic Anaplasma agent, in acute phase blood samples of naturally infected humans or animals or experimentally-infected animals such as horses, are co-incubated with log phase growth of promyelocytic leukemia cells, such as HL60 or KG-1 cells.

Illustratively, KG-1 cells are preferably cultured in suspension in a tissue culture medium, such as RPMI 1640 supplemented with 5.0 to 20% (v/v), preferably about 10% (v/v), heat-inactivated fetal bovine serum and 2.0 mM·L-glutamine and in an atmosphere of 5% CO₂ at about 37° C. Antibiotics, such as penicillin with streptomycin, or gentamicin can be added for maintenance cultures to suppress bacterial contamination, as is standard in the art. The doubling time of the cultures under these conditions is approximately 48 to 72 hours, and thus cell concentration should be carefully maintained, preferably below 1.0×10⁷ cells per ml of tissue culture medium. Thus, log phase growth HL60 or KG-1 cells are centrifuged and resuspended in tissue culture medium at a concentration of about 3.0×10⁶ cells per ml. Cell culture medium is preferably partially replaced 2 to 3 times per week with freshly prepared medium.

The HL60 (ATCC No. CCL 240) and KG-1 (ATCC No. CCL 246) human promyelocytic leukemia cell lines are propagated in RPMI 1640 medium with 10 to 20% (v/v) fetal bovine serum, 2.0 mM L-glutamine, with or without penicillin and streptomycin. Log phase HL60 or KG-1 cells are centrifuged, counted, and resuspended into fresh medium without penicillin and streptomycin at a cell density of about 3.0×10⁶ cells per ml. Whole human or equine blood, anticoagulated with ethylene diaminotetraacetic acid (EDTA) or acid citrate dextrose (ACD), known to be infected with the human granulocytic Anaplasma or A. equi, of which less than 10% of human peripheral blood neutrophils contained Anaplasma morulae and less than 50% of equine peripheral blood neutrophils contained A. equi or human granulocytic Anaplasma morulae, are used.

Between 300 to 500 l of this infected blood are added directly into 25 cm² plastic tissue culture flasks containing 5.0 to 9.0 ml of HL60 or KG-1 cells. Similarly, an equivalent inoculum of uninfected human or equid blood, or blood from a patient recovering from human granulocytic Anaplasmosis after 2 to 3 days of doxycycline therapy is incubated with 5.0 to 9.0 ml of HL60 or KG-1 cells, and 5.0 to 9.0 ml volumes of HL60 cells are held as uninoculated controls. The flasks are maintained in an atmosphere of 5% CO₂ at 37° C.

Cell cultures are examined every two to four days by removing small aliquots and preparing cytocentrifuged slides that were then examined microscopically after Romanowsky (LeukoStat®, Fisher Scientific, Pittsburgh, Pa.) staining. Aliquots of the same cells are stored for later examination to detect the presence of Anaplasmae species and Ap group granulocytic Anaplasmae DNA.

Before day 3, control HL60 and KG-1 cells, control HL60 and KG-1 cells inoculated with uninfected blood, blood from a doxycycline-treated patient recovering from human granulocytic Anaplasmosis, and HL60 cells inoculated with A. equi-infected equid blood appear identical, except for the presence of occasional normal blood erythrocytes and leukocytes in the blood-inoculated cultures among the HL60 or KG-1 leukemia cells. By 3 days after inoculation, typical intracytoplasmic inclusions (morulae) filled with individual bacterial bodies (Anaplasmae) are present within vacuoles of 3.0% of cells in the flask of KG-1 cells inoculated with human granulocytic Anaplasma-infected equid blood. By 4 days after inoculation, typical intracytoplasmic inclusions (morulae) filled with individual bacterial bodies (Anaplasmae) are present within vacuoles of 12.0% of cells in the flask of HL60 cells inoculated with A. equi-infected equid blood, and by 5 to 9 days after inoculation, typical intracytoplasmic inclusions (morulae) filled with individual bacterial bodies (Anaplasmae) are present within vacuoles of 21.0 to 31.0% of cells in the flask of HL60 cells inoculated with human granulocytic Anaplasma-infected human blood.

No morulae are noted in any of the control HL60 or KG-1 flasks. Between 2 to 3 weeks after inoculation of cultures with infected blood, morulae are present in from zero to 100% of cells, and in many cases, multiple, complex morulae in various stages from discrete organisms to large aggregates, and in some instances appearing to cause cell lysis and subsequent extracellular release of organisms. Control cells contain no such structures. The morphology of many of these morulae, especially in the early cultures, is quite similar to those seen in the peripheral blood neutrophils in human granulocytic Anaplasmosis in that these appear to have generally even, rounded contours within the apparent cytoplasmic vacuoles.

The co-cultivated cells are maintained in suspension cultures and are examined periodically, preferably weekly, by Romanowsky staining or immunofluorescence, for the presence of typical Ap genogroup Anaplasma species morulae and antigens indicative of infection. After about ten days, morulae are detectable by Romanowsky staining, and by day 21, more than 50% of the HL60 cells contain Anaplasma species morulae. The infected HL60 or KG-1 cells can then be harvested and used for antigen or nucleic acid preparation.

The above-prepared A. equi-infected IDE8 cells can also be used to infect human promyelocytic leukemia cell lines. For example, A. equi-infected IDE8 cells are mixed with the HL60 cells, preferably at a ratio of approximately 1:36 (0.25 ml of infected tick cells and 9.0 ml of HL60 cells). The preferred culture conditions include RPMI 1640 medium supplemented with about 10% (v/v) fetal bovine serum (heat-inactivated), about 2.0 mM L-glutamine, and in an atmosphere of 5% CO₂ at about 37° C. The co-cultivated cells and HL60 cells are maintained in suspension cultures and are examined periodically, preferably weekly, by Romanowsky staining, for the presence of typical Anaplasmae morulae indicative of infection.

Infected cells can contain single morulae typical in appearance, as seen in the peripheral blood neutrophils of patients with human granulocytic Anaplasmosis, and horses or dogs with A. equi granulocytic Anaplasmosis. With progressive time, an increasing percentage of cells become infected, and may contain multiple morulae, providing an in vitro method to obtain substantial quantities of Ap group Anaplasmae propagated within mammalian (human) cells.

For assay purposes, it is preferred that about 50-100% of the HL60 cells are infected with Anaplasma phagocytophilia or Anaplasma equi. Illustrative immunoassays are often carried out on a microscope slide. Here, about 100 to about 1000 HL60 or other promyelocytic cells are present on a slide, and no less than about 20% of the cells are infected with Anaplasma phagocytophilia or equi.

Growth and development of A. equi or the human granulocytic Anaplasma agent in, e.g., HL60 or KG-1 cells can be detected by several methods. One method is to prepare cell spreads using a cytocentrifuge and to stain the cells with a Romanowsky stain. Infected cells can then be detected by the presence of Anaplasmae inclusions in the cytoplasm. The inclusions seen in HL60 and KG-1 cells are similar to those seen in vivo in the neutrophilic granulocytes of humans, dogs and horses. Inclusions in heavily infected cells can completely fill the cytoplasm, and cause the cell to distend and rupture.

Alternatively, growth can be detected by immunocytological methods using specific A. equi, A. phagocytophila, or HGA agent antibodies in indirect fluorescent antibody methods and indirect immunoenzymatic methods.

Promyelocytic leukemia cells, such as HL60 and KG-1 cells, infected with A. equi or the human granulocytic Anaplasma agent or cell cultures are also useful as diagnostic tools for assays including ELISA, indirect fluorescent antibody tests, latex agglutination tests, complement fixation tests, and immunoblot tests. For immunofluorescence, it is preferred that 100 to 1000 cells be present on a slide with about 50% to 100%, and no less than 25% of cells infected.

### Example 2

### Illustrative PCR

The identity of A. equi is confirmed using a DNA oligonucleotide primers SLE1-F (SEQ ID NO:1); and SLE1-R (SEQ ID NO:2), by a standard PCR. The antigenic identity of the A. equi in IDE8 tick cultures is also confirmed by an immunocytology using polyclonal horse anti-A. equi and polyclonal human anti-human granulocytic ehrlichiosis agent antibodies.

PCR using infected tick cell culture extract as a template confirms the identity of the A. equi growing in IDE8 cells. A crude lysate is made according to rapid sample preparation for PCR [Higuchi, In: PCR Technology, Principles and Applications for DNA Amplification, H. A. Ehrlich, Ed. Stockton Press, New York, Chapter 4 (1989)].

Briefly, infected tick cells from one culture are forced about 10 times through a 27 gauge needle, and large debris removed by centrifugation at 100×g. The supernatant fluid containing small particles and Anaplasmae are collected by centrifugation at 10,000×.g for 20 minutes, and the pellet is resuspended in lysis buffer with NP-40, Tween®-20 and Proteinase K (Higuchi, supra). Following incubation (maintenance) at 55° C. for 1 hour, the proteinase is inactivated (95° C. for 20 minutes), and the lysate stored at −20° C. Uninfected IDE8 cells are extracted the same way as a control.

Ten μl of that lysate are used as a template in the PCR with primers SLE1-F and SLE1-R at a concentration of 0.5 μM each. 100 μl reaction mixtures containing 1.5 mM MgCl₂ and 0.2 mM of the four deoxynucleotides are cycled 30 times through 92° C. for 1 minute, 56° C. for 0.5 minutes and 72° C. for 1 minute. Ten μl of the resulting DNA is mixed with loading buffer (Ficoll® 400 with bromophenol blue), and electrophoresed through 0.9% (w/v) agarose in 0.5×TBE (Tris-Borate-EDTA) buffer at 150 volts until adequately separated.

The gel is stained with ethidium bromide and photographed under UV light. Lysate from infected IDE8 cultures gives rise to a DNA product of the expected size, 919 bp, whereas control lysate does not, indicating the presence of A. equi DNA and thus, A. equi in the infected cultures. A plasmid encoding the entire A. equi 16S ribosomal RNA gene is used as positive control and produces an identical 919 bp band, whereas a negative control using no template produces no detectable band.

### Example 3

### Bladder Cancer PCR

A 52 year old male patient presented with a stage iv bladder cancer. Three tubes of blood were obtained-2 with citrate (run in duplicate lanes 2/3, 4/5) and one without anticoagulant (lanes 6/7). Plasmid controls, run at 3 dilutions are in lanes 8-10 and the negative control was run in lane 11.

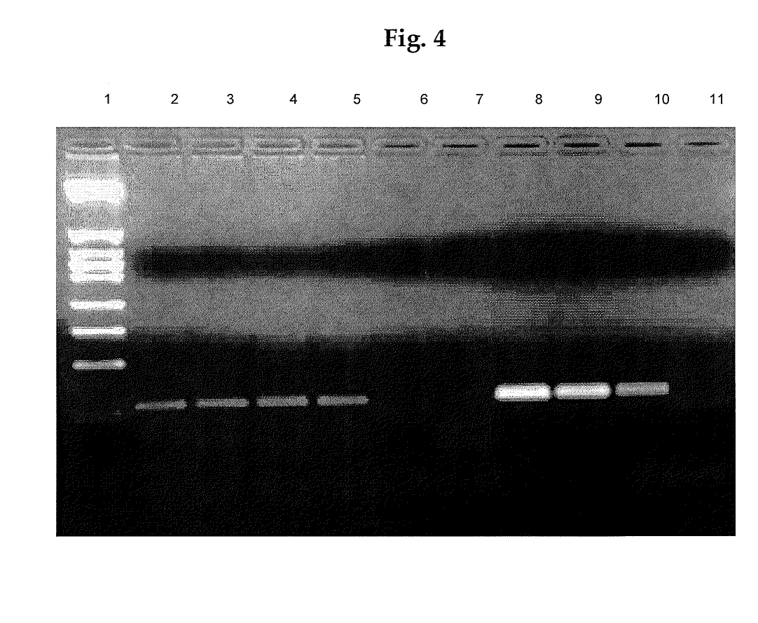

Three rounds of PCR were carried out by Medical Diagnostic Laboratories, L.L.C. of Hamilton, N.J. using their own primers. The resulting amplified nucleic acid was run on a gel as seen in FIG. 4. The results show that there are low level bands that are amplified above and below the expected fragment size.

Also, the bands that seen in the lanes representing the patient's samples are a little different from the controls based upon the melt curve data obtained following the PCR amplification. It appears as if whatever is amplifying in this patient's blood is slightly different, sequence-wise, from the control sequence, and melts 2 degrees lower. Nonetheless, these results indicate that a bacterium very similar to HGA is present in the patient's blood.

### Example 4

### Patients

Patient AM

This was a 64-year-old male with acute lymphocytic leukemia (ALL), who asked for help when he had an untreatable recurrence after a marrow transplant, and was in end stage marrow failure. The patient, who could not sit up in bed, asked for and received treatment with the antibiotic Rifampin and had a remarkable though temporary recovery. No other treatment was available or given. After 2 months of rapid improvement, during which he regained the ability to walk and was discharged from the hospital, he succumbed to an acute apparently unrecognized cytomegalovirus infection.

Patient MR

This was a 14 year old boy with acute myelogenous leukemia (AML), who had blood counts of less than 2000 platelets and less than 300 white cells, persisting for 3 months. He was treated with Rifampin and on day 11 his white cell count increased to 21,000, with 75% blasts. Platelets increased less rapidly, however, and despite significant improvements in other laboratory values, he suffered sudden hemorrhagic phenomena and died of blood loss.

Patient AP

This patient had been treated by splenectomy for polycythemia vera (PV). Significant microscopic evidence of Ap was found in her erythrocytes, platelets and leukocytes. PCR analysis revealed evidence of the major surface protein of Anaplasma phagocytophylium. She suddenly converted to AML, chose no treatment, and expired within 2 weeks.

### Example 5

### Genomic Studies

Genomic studies were carried out on the blood of Patient AP. Those studies indicated a single residue difference at sequence position 591 between the sequence of the major surface protein of A. phagocytophylium (APC) and the product of the PCR from the blood of patient AP. Additionally the blood of this patient reacted repetitively positive to a probe for HGA (human granulocytic Anaplasma) (18/18). Microscopic evidence was shown of organisms resembling A. phagocytophylium in leukocytes, platelets, and erythrocytes. Alignment of the sequences obtained by PCR with the deposited sequence of A. phagocytophylium were within 96% of the sequences of the major surface protein 2 of APC.

More specifically, total DNA was extracted from a 200 ml fraction of patient blood using the QIAamp® DNA Blood Mini Kit (Qiagen, Valencia, Calif.). Following PCR amplification with an in house-developed assay for human granulocytic Anaplasmosis (HGA), the amplicon was cloned into the TOPO® TA cloning vector (Invitrogen Co., Carslbad, Calif.). Colonies were selected based on a blue-white screen and verified directly by PCR using the HGA assay. Mini preparations of DNA were prepared from small cultures of PCR-confirmed positive colonies; successful cloning of the amplicon was further confirmed through plasmid digestion with EcoRI and gel electrophoresis.

PCR reactions utilizing SYBR® Green intercalating dye were subsequently run and the resulting product used for DNA sequence analyses (Genetic Analyzer 3130, ABI) in both forward and reverse orientations. Approximately 70 base pairs were identified from two independent plasmids.

Obtained sequences were submitted for nucleotide BLAST (Basic Local Alignment Tool Analysis, v 2.2.18) analyses using the BLASTN version 2.2.18+ database (blast.ncbi.nlm.nih.gov/Blast.cgi) for identification. The sequence of the clinical unknown was compared against all Genbank, EMBL, DDBJ, and PDB deposited sequences, which combined total of 7,064,549 sequences. The leading 100 matching sequences aligned with a maximum identity of 96% or greater to A. phagocytophylium major surface protein 2 from various deposited strains and E-values (Expect values) of 1e⁻⁴⁷ or lower.

Sybr 8_{—}5R.seq: DNA preparation 8 (5 ml) with the reverse primer as the sequencing primer.

Sybr 1_{—}5R.seq: DNA preparation 1 (5 ml) with the reverse primer as the sequencing primer.

Sybr 1_{—}5F.seq: DNA preparation 1 (5 ml)) with the forward primer as the sequencing primer.

Sybr 8_{—}5F.seq: DNA preparation 8 (5 ml) with the forward primer as the sequencing primer.

Sybr 8 F.seq: DNA preparation 8 (1 ml) with the forward primer as the sequencing primer.

Sybr 1 F.seq: DNA preparation 1 (1 ml) with the forward primer as the sequencing primer.

HGE amplicon.seq: represents the deposited sequence for A. phagocytophylium amplified with the reverse primer.

SEQ ID NO: 10
TTTCGTAACC AATCTCAAGC TCAACCCWGG CACCACCAAT
ACCATAACCA ACACTGCCTT CCATAGCTAC AAGCATGTTG
TCCTTAAACC CAATCCGAGG ATCAGGAGTG TTCCAGTCAA
AGGGCGAATT CCAGCACAC
HGA
SEQ ID NO: 12
TCGTAACC AATCTCAAGC TCAACCCTGG CACCACCAAT
ACCATAACCA ACACTGCCTT CCATAGCTAC AAGCATGTTG
TCCTTAAACC CAATCCGAGG ATCAGGAGTG TTCCAGTCA
Primer
(SEQ ID NO: 11)
5′-TCCTCGGATTGGGTTTAAGGA

An annealing temperature of 62° C. was used for these studies.

### Example 6

### Human Aplastic Anemia Associated with Ap

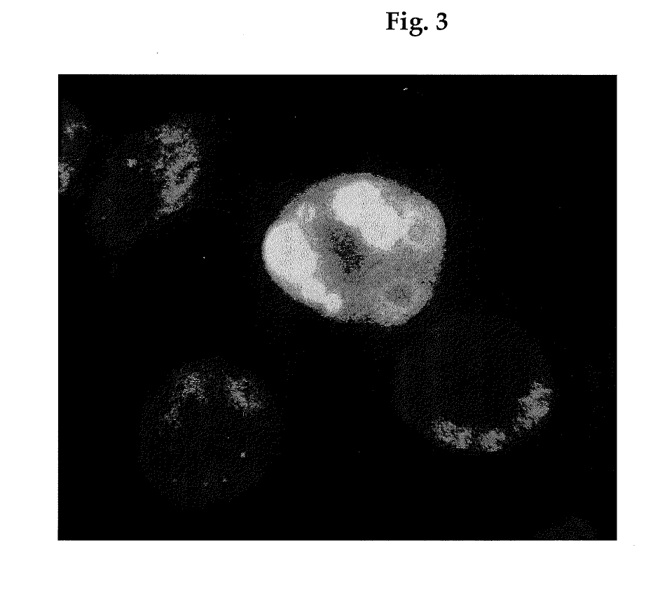

The first report of an Ap in a case of human aplastic anemia was reported in an abstract in 1973. Dr. M. Nyindo in collaboration with the present inventor [lead author of the abstract] was able to culture the organism. Infected monocytes were observed in a Rhesus monkey (FIG. 3) after injection of the animal with supernatant fluid from the culture of the human aplastic anemia. The infection was presumably caused by initial bodies of Ap present in the supernatant fluid from the culture.

The organism was successfully transmitted to a second Rhesus monkey. Thus, an Ap organism from a human with aplastic anemia biologically and antigenically related to the Anaplasma was cultured from a patient with a myelodysplastic syndrome, and successfully transmitted sequentially to a first and then a second primate [Kallick et al., “Human Bone marrow failure associated with Ehrlichia canis”, Presented at the Interscience Conference on Antimicrobial Agents and Chemotherapy, Washington D.C., 1973, Abstract #1]. These data strongly suggest that Ap are infective agents for canines and humans.

Each of the patents and articles cited herein is incorporated by reference. The use of the article “a” or “an” is intended to include one or more.

The foregoing description and the examples are intended as illustrative and are not to be taken as limiting. Still other variations within the spirit and scope of this invention are possible and will readily present themselves to those skilled in the art.

What is claimed:

1. A method of assaying for the presence of Anaplasma-infected blood cells or blood cell precursors from a patient that comprises
a) amplifying a nucleic acid-containing preparation of blood cells or blood precursor cells with a primer that contains a sequence of at least 12 consecutive bases of a polynucleotide of SEQ ID NOs:1-16 or an antisense sequence thereof; and
b) detecting the presence of an amplified nucleic acid sequence, wherein the presence of an amplified sequence indicates that the patient is suffering from Anaplasma-infected blood cells or blood cell precursors.

2. The method according to claim 1, wherein said primer is double-stranded.

3. The method according to claim 1, wherein said primer contains a sequence of about 20 to about 30 consecutive bases of a polynucleotide of SEQ ID NOs:1-16 or an antisense sequence thereof.

4. The method according to claim 1, wherein the patient exhibits symptoms of systemic lupus erythematosus, aplastic anemia, bladder cancer, or leukemia.

5. The method according to claim 1, wherein the patient exhibits symptoms of a leukemia that is acute lymphocytic leukemia, acute myelogenous leukemia, polycythemia vera or chronic lymphoid

6. The method according to claim 1, wherein said primer contains a sequence of about 20 to about 40 consecutive bases of a polynucleotide of SEQ ID NOs:10-16 or an antisense sequence thereof.

7. A kit for determining the presence of blood cells or blood precursor cells infected with Anaplasma that comprise one or more containers that hold a primer that contains a sequence of at least 12 consecutive bases of a polynucleotide of SEQ ID NOs:1-16 or an antisense sequence thereof.

8. The kit according to claim 7 that further includes a second container of primer such that a first set of primers that amplify a sequence of about 100 bp and second pair of primers that amplify a DNA sequence of 75 bp are present in said containers.

9. The kit according to claim 7, wherein said primer is double-stranded.

10. The kit according to claim 7, wherein said primer contains a sequence of about 20 to about 30 consecutive bases of a polynucleotide of SEQ ID NOs:1-16 or an antisense sequence thereof.

11. The kit according to claim 7, wherein said primer contains a sequence of about 20 to about 40 consecutive bases of a polynucleotide of SEQ ID NOs:10-16 or an antisense sequence thereof.


SEQUENCE LISTING (17 sequences)

SEQ ID NO: 1 - DNA, 25 bp, Artificial sequence
  synthetic sequence
aacggattattctttatagcttgct

SEQ ID NO: 2 - DNA, 25 bp, Artificial sequence
  synthetic sequence
ggagattagatccttcttaacggaa

SEQ ID NO: 3 - DNA, 28 bp, Artificial sequence
  synthetic sequence
gtcgaacggattattctttatagcttgc

SEQ ID NO: 4 - DNA, 28 bp, Artificial sequence
  synthetic sequence
cccttccgttaagaaggatctaatctcc

SEQ ID NO: 5 - DNA, 117 bp, Artificial sequence
  synthetic sequence
tcgtaaccaatctcaagctcaaccctggcaccaccaataccataaccaacactgccttccatagctacaagcatgttgtccttaaacccaatccgaggatcaggagtgttccagtca

SEQ ID NO: 6 - DNA, 24 bp, Artificial sequence
  synthetic sequence
tagatccttcttaacggaagggcg

SEQ ID NO: 7 - DNA, 24 bp, Artificial sequence
  synthetic sequence
aagtgcccggcttaacccgctggc

SEQ ID NO: 8 - DNA, 25 bp, Artificial sequence
  synthetic sequence
tttatcgctattagatgagcctatg

SEQ ID NO: 9 - DNA, 23 bp, Artificial sequence
  synthetic sequence
ctctacactaggaattccgctat

SEQ ID NO: 10 - DNA, 139 bp, Artificial sequence
  synthetic sequence
tttcgtaaccaatctcaagctcaacccwggcaccaccaataccataaccaacactgccttccatagctacaagcatgttgtccttaaacccaatccgaggatcaggagtgttccagtcaaagggcgaattccagcacac

SEQ ID NO: 11 - DNA, 21 bp, Artificial sequence
  synthetic sequence
tcctcggattgggtttaagga

SEQ ID NO: 12 - DNA, 20 bp, Artificial sequence
  synthetic sequence
gcttgagattggttacgaaa

SEQ ID NO: 13 - DNA, 25 bp, Artificial sequence
  synthetic sequence
cctgggttgagcttgagattggtta

SEQ ID NO: 14 - DNA, 25 bp, Artificial sequence
  synthetic sequence
ccagggttgagcttgagattggtta

SEQ ID NO: 15 - DNA, 19 bp, Artificial sequence
  synthetic sequence
tgactggaacactcctgat

SEQ ID NO: 16 - DNA, 40 bp, Artificial sequence
  synthetic sequence
cctcggattgggtttaaggacaacatgcttgtagctatgg

SEQ ID NO: 17 - DNA, 117 bp, Artificial sequence; identical to SEQ ID NO: 5
  synthetic sequence